(19) United States
(12) Patent Application Publication
(10) Pub. No.: US 2023/0298760 A1
(43) Pub. Date: Sep. 21, 2023

(54) SYSTEMS, DEVICES, AND METHODS FOR DETERMINING MOVEMENT VARIABILITY, ILLNESS AND INJURY PREDICTION AND RECOVERY READINESS

(71) Applicant: SPARTA SOFTWARE CORPORATION, Menlo Park, CA (US)

(72) Inventor: Phillip Patrick Wagner, Menlo Park, CA (US)

(21) Appl. No.: 17/978,360

(22) Filed: Nov. 1, 2022

Related U.S. Application Data
(63) Continuation of application No. PCT/US21/30596, filed on May 4, 2021.
(60) Provisional application No. 63/020,349, filed on May 5, 2020.

(51) Int. Cl. G16H 50/30; G16H 50/20; A61B 5/00; A61B 5/103

(52) U.S. Cl. CPC G16H 50/30; G16H 50/20; A61B 5/7275; A61B 5/4023; A61B 5/1036

(57) ABSTRACT

Systems, devices and methods are provided for determining injury risk and athletic readiness based on user movement data, including movement variability data. Generally, a sensor device, such as a force plate, is provided for sensing certain characteristics of a user movement. A computing device coupled to the sensor device can be configured to receive sensor data indicative of the characteristics of the user movement, process and extract information from the sensor data, and transmit the processed sensor data to a server system. The remote server system can be configured to store, aggregate and update the processed sensor data in a database, and can also generate one or more normalized scores correlating to the user movement. The normalized scores can indicate to a user a susceptibility to injury and/or a readiness towards return to normal activity.

### CROSS-REFERENCE TO RELATED APPLICATIONS

The subject application is a continuation of International Patent Application No. PCT/US21/30596, filed May 4, 2021, which claims priority to U.S. Provisional Patent Application No. 63/020,349, filed on May 5, 2020, both of which are incorporated by reference herein in their entirety for all purposes.

### FIELD

The subject matter described herein relates generally to systems, devices, and methods for determining movement variability, illness and injury prediction and recovery readiness based at least in part on postural balance data. In particular, sensor data for a user performing one or more postural balance movements is captured by one or more sensors, analyzed, and transformed into one or more scores using a population database, or a subset thereof.

### BACKGROUND

Advances in kinesiology and sensor technology have enabled researchers to capture and study vast amounts of data regarding the human body during movement, and in particular, human movement variability. Clinical tests such as the Balance Error Scoring System (BESS) and King-Devick (KD) are utilized frequently in the athletic population for baseline testing, and additionally as return to play criteria. However, the sensitivity of the BESS has been questioned, as the distribution of scores for an uninjured population demonstrates a floor effect, and substantial inter-rater variability exists. Biomechanical testing is able to detect smaller magnitudes of change but has not been validated against these clinical tests.

Performance of human daily activities is based on simple movements such as standing or walking. However, these tasks require complex control mechanisms, including those of the lower body extremities. Movement variability is a key factor in performance and well-being, from the elderly population to professional athletes. It is known that movement variability plays a key role in the detection and exploration of stability boundaries during balance control. Movement variability is not necessarily disadvantageous. In fact, common transitions in movement behavior (standing to walking, walking to sitting, etc.) require increased variability in order to optimize coordination. However, human movement variability is a complex system with optimal ranges, bookended by ranges that will detrimentally affect an individual, either with too much variability or not enough. Currently there is no known simple way to quantify human movement variability of balance tests in order to create optimal ranges for specific populations. These optimal ranges could be used in illness and injury prediction and recovery tracking.

Thus, needs exist for systems, devices and methods for objectively determining movement variability, illness and injury prediction and readiness recovery based at least in part on using simple manner of postural balance data to quantify human movement variability of balance tests and to create optimal ranges for specific populations. The tests may utilize a software platform based around a force plate.

### SUMMARY

Provided herein are example embodiments of systems, devices and methods for determining movement variability, illness and injury prediction and recovery readiness based at least in part on postural balance data. In particular, sensor data for a user performing one or more postural balance movements may be captured by one or more sensors, analyzed, and transformed into one or more scores or vital signs (for example, similar to blood pressure and heart rate) using a population database, or a subset thereof. In some embodiments, four stages of testing may be performed and may include double-leg eyes open balance, double-leg eyes closed balance, single-leg eyes open balance, and single-leg eyes closed balance. In some embodiments, the disclosed systems, devices and methods may generally include the ability to quantify in a simple manner, using a software platform based around a force plate, human movement variability during a series of balance tests. The software may then generate population-specific optimal ranges which may be used to guide training, monitor care, health and well-being of the individual, tracking progress or decline. The present methods of measuring movement variability advantageously surpass other methods by considering more directional data, as well as more testing conditions, and finally the ability to track progress or health versus a population-specific database.

Generally, a computing device is provided, where the computing device is communicatively coupled to one or more sensors that are adapted to sense various characteristics of one or more human movements. For example, the computing device may be a local device coupled to the one or more sensors. The characteristics can include, for example, a plurality of ground reaction forces. The one or more sensors can be included within a single housing, such as that of a force plate. The computing device, to which the one or more sensors are coupled, can also include, amongst other components, communications circuitry, one or more processors and a memory coupled to the one or more processors. The memory is configured to store instructions that, when executed by the one or more processors, cause the one or more processors to perform various method steps for determining movement variability, illness and injury risk and recovery readiness. For example, the computing device can be configured to receive and process sensor data indicative of the various characteristics of the one or more human movements, and in turn, transmit the processed sensor data to a server system, which may, for example, be a remote server system. The computing device can also be configured to process, update, and transmit one or more user profiles to the server system. The user profiles can include data associated with dates, times, and frequency of the one or more human movement assessments for one or more associated users.

In some embodiments, a server system, which may, for example, be a remote server system, is provided for receiving and storing the processed sensor data and user profile data, and can also be configured for transmitting back to the local computing device one or more scores, including, for example, movement variability scores, illness and injury risk scores, readiness scores, and other normalized scores correlating to the processed sensor data associated with the one or more human movements. In some embodiments, multiple variables may be analyzed including resultant sway velocity, anterior-posterior (AP) sway velocity, medial-lateral (ML) sway velocity, and sway velocity frequencies in both the AP and ML directions. In some embodiments, the illness and injury risk may include risk of concussion, for example, from falling.

The movement variability score and illness and injury risk score can be based at least in part on one or more differential values of the processed sensor data. The readiness score can be based at least in part on the movement variability score and illness and injury risk score and a frequency of human movement assessments. The scores can be T-scores, which can be normalized by various factors, such as by body weight, by gender, by movement variability or by illness and injury risk. When users are athletes the T-scores can also be normalized by preferred sport and by preferred position within a sport. In addition, the remote server system can include, or be communicatively coupled to, a database comprising stored processed sensor data indicative of characteristics of various human movements for a defined population and user profiles. In this regard, and as described in further detail below, the movement variability scores, illness and injury risk scores, readiness scores, and normalized scores can provide a variety of objective and actionable data to a healthcare provider, a coach, a manager, a patient or an athlete, such as, for example, susceptibility to fall, injury, progression towards return to previous activities, suitability for a particular sport, and/or readiness to perform a particular activity or play a sport at a given time, to name a few. Readiness can include at least the individual's availability and ability to perform the activities.

In some embodiments, the variables disclosed herein may include one or more characterizing features. The variables may include, for example, illness and injury risk, and movement variability.

In some embodiments, the system may process time series data from the assessments, or scans, and extract one or more set of features. The features may be extracted through a variety of techniques and may be used as inputs for machine learning models.

In some embodiments, the system may include a trained injury risk model to predict relative injury risk for individuals for a given set of features extracted from scans. The models may be trained using injury event data associated with periods with which scan data is associated.

To ensure the integrity of the acquired and processed sensor data, several data validation methods are also provided. For example, in some embodiments, prior to the user performing movement variability tests, the measured weight of the user is compared to a stored reference weight. If a weight mismatch is detected, e.g., if the measured weight is inaccurate or the user has misidentified herself, then the user is instructed to weigh in again. In other embodiments, one or more predetermined weight thresholds are monitored during the user movement which can detect, for example, a user prematurely stepping off the force plate, or the user not standing still on the force plate with sufficient time. In still other embodiments, a final data check is performed before the processed sensor data is transmitted to the remote server system, which can be used to detect a corrupt file. These and other data validation systems and methods are described in U.S. Pat. Nos. 9,682,280, 9,737,758, 9,223,855, 10,471,290, as well as PCT Patent Application Nos. PCT/US18/40687, PCT/US18/55163, and PCT/US19/35361, which are incorporated by reference in their entirety for all purposes.

In some embodiments, sensors used may also include accelerometers, inertial sensors, heart rate sensors, etc., to name a few.

In some exemplary applications, the present disclosure may be used in monitoring someone suffering after a stroke. Strokes typically cause unilateral weakness and poor coordination. If someone were to suffer from a stroke, they would initially be tested using the double-leg eyes open condition of the balance test. The test would include, for example, four trials, 20 seconds each. Using a database of healthy and stroke-specific populations, the individual's scores may be compared to determine if their movement variability was too high, too low, or within an acceptable range. The patient may be tested each day to track progress and determine if the patient has recovered or requires additional treatment. When the scores are within a normal, acceptable range for a certain testing condition (e.g., double-leg eyes open), the patient may progress to the next testing conditions (e.g., double-leg eyes closed, single-leg eyes open, single-leg eyes closed). The variables of interest may stay the same across testing conditions and each condition may have population-specific ideal ranges. Patients may be supported through training protocols so as to optimize dynamic control and variability improvement.

In some other exemplary applications, the present disclosure may be used to detect concussion and monitor recovery, such as those systems, methods, and devices described in Appendix A, which is attached hereto and hereby incorporated by reference in its entirety for all purposes. Other applications may include pediatric growth monitoring, prediction and rehabilitation monitoring of concussion, lower leg injuries, knee injuries, to name a few.

In some exemplary applications, the present disclosure may be used to treat populations with neurological conditions (e.g., in doctor's office, assisted living facility, rehabilitation site, etc.).

These embodiments and others described herein are improvements in the fields of computer-assisted biomechanics and, in particular, in the area of computer-based kinetic and kinematic analysis. Other systems, devices, methods, features and advantages of the subject matter described herein will be apparent to one with skill in the art upon examination of the following figures and detailed description. The various configurations of these devices are described by way of the embodiments which are only examples. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of one or more local computing devices each of which can be coupled to a sensor device, a network, and a remote server system including a database.

FIG. 2 is a block diagram of an example embodiment of a local computing device.

FIG. 3A is a block diagram of an example embodiment of a remote server system.

FIG. 4A is a flow chart diagram depicting example embodiment methods for assessing a user's illness and injury risk score.

FIGS. 4B to 4I are pictorial diagrams depicting certain steps in the example embodiment method of FIG. 4A.

FIG. 5 is an exemplary graphical user interface illustrating calculated metrics for a user.

### DETAILED DESCRIPTION

Before the present subject matter is described in detail, it is to be understood that this disclosure is not limited to the particular embodiments described herein, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

Generally, embodiments of the present disclosure include systems, devices, and methods for determining movement variability, illness and injury prediction and recovery readiness based at least in part on postural balance data. In some embodiments, recovery readiness may include readiness to resume human tasks prior to an accident, including walking, running, playing in a sport, etc. Accordingly, many embodiments can include one or more sensor devices coupled to one or more local computing devices, wherein the one or more sensor devices are configured to measure various characteristics of a human movement performed by a user. In addition, many embodiments can include a remote server system which can include, or be communicatively coupled with, a database configured to store processed sensor data associated with various user movements for a population of users.

In some embodiments, for example, a force plate can be configured to measure a resultant sway velocity and velocity frequencies associated with a user standing in a balance pose on the force plate. As described in further detail herein, the user may be standing in various manners, e.g., double-leg eyes open, double-leg eyes closed, single-leg eyes open, single-leg eyes closed. The resultant sway velocity and velocity frequencies are transmitted to a remote server system, and, subsequently, one or more normalized scores correlating to the resultant sway velocity and velocity frequencies are displayed on the local computing device. In these embodiments, the normalized scores can reflect a user's movement variability and balance stability.

In other embodiments, a force plate can be configured to measure movement variability and balance stability and time to stabilize within a predetermined percentage of a reference weight.

In some embodiments, the remote server system may include complex statistical analysis using machine learning, for example, to predict injuries, for example, concussion, to improve rehabilitation treatments, and to predict and determine recovery readiness, to name a few.

Additionally, the present disclosure also includes systems and methods for validating the data acquired by the one or more sensors, and can include, for example, a weight mismatch process, a weight deviation process, sway velocity and velocity frequencies process, a premature end condition monitoring process, and a final data check process, among others, each of which is described in further detail below. The embodiments disclosed herein can include local computing devices, each of which is in communication with a remote server system that is location-independent, i.e., cloud-based. Those of skill in the art will also appreciate that the embodiments disclosed herein can also include local computing devices, each of which is in communication with a remote server system that is located on the same premise and/or local area network as the one or more local computing devices. In these embodiments, the remote server systems which are located on the same premise and/or local area network as the one or more local computing devices can also be configured to synchronize a database containing processed sensor data associated with a population of patients and athletes with a database residing on, or coupled with, a centralized remote server system that is location-independent, i.e., cloud-based.

Furthermore, for each and every embodiment of a method disclosed herein, systems and devices capable of performing each of those embodiments are covered within the scope of the present disclosure. For example, embodiments of sensor devices, local computing devices, and remote server systems are disclosed, and these devices and systems can each have one or more sensors, analog-to-digital converters, one or more processors, memory for storing instructions, displays, storage devices, communications circuitries (for wired and/or wireless communications), and/or power sources, that can perform any and all method steps, or facilitate the execution of any and all method steps.

The embodiments of the present disclosure provide for improvements over prior modes in the field of computer-based kinetic and kinematic analysis. These improvements can include, for example, optimization of computer resources, improved data accuracy and improved data integrity, to name only a few. In a number of embodiments, for example, instructions stored in the memory of a local computing device (e.g., software) can cause one or more processors of the local computing device to process and extract certain characteristics from sensor data associated with one or more user movements received from a sensor device (e.g., a force plate), and transmit the processed sensor data to a remote server system. Subsequently, the remote server system receives and stores the processed sensor data, and returns to the local computing device one or more normalized scores correlating to the user movement. The normalized scores can be T-scores, for example, and displayed on the local computing device in an easy-to-read format, e.g., vertical bar chart. The sensor data on the local computing device can be subsequently discarded. Thus, according to one aspect of the embodiments, memory and hard drive space are conserved at the local computing device because sensor data need not be permanently stored. Likewise, the remote server system need only store processed sensor data (i.e., extracted values), and is not required to process or store raw sensor data, thereby conserving memory, hard drive space and processing power. Thus, computer resources can be significantly conserved both at the local computing device as well as at the remote server system.

The disclosed embodiments also reflect computer-related improvements in data accuracy and data integrity. In some embodiments, for example, the remote server system includes, or is communicatively coupled with a database for storing processed sensor data correlating to a population of patients and athletes. According to one aspect of the disclosed embodiments, the remote server system can be location-independent (i.e., cloud-based), and configured to aggregate processed sensor data from a plurality of local computing devices, which can be located in a plurality of geographically dispersed areas. The remote server system can also provide normalized scores to each local computing system based on the population data contained in the database. The normalized scores can also be normalized according to categories, for example, by gender, by body weight, by sport or by position within a sport. By continually aggregating and updating the population data contained within the database, the remote server system can be configured to provide customizable, dynamically generated and accurate scores to the user.

According to another aspect of the disclosed embodiments, improvements in data integrity are also provided through data validation processes during the acquisition of the sensor data. As described in further detail below, the data validation processes can include, for example, a weight mismatch process, a weight deviation process, sway velocity and velocity frequencies process, a premature end condition monitoring process, a weight validation process, a minimum velocity process, a minimum velocity frequency process, and a final data check process, among others. Each of these processes, as well as others, are configured to ensure that the acquired sensor data is accurate prior to processing and receiving the processed sensor data by the remote server system. Other sensor data validation processes are described in U.S. patent application Ser. No. 62/528,866, which is incorporated by reference in its entirety for all purposes.

The improvements of the present disclosure are necessarily rooted in computer-based systems for determining movement variability, illness and injury risk and recovery readiness based on human movement data, and are directed to solving a technological challenge that might otherwise not exist but for the existence of computer-based kinetic and kinematic analyses. Additionally, many of the embodiments disclosed herein reflect an inventive concept in the particular arrangement and combination of the devices, components and method steps utilized for acquiring, validating and analyzing user movement data. Other features and advantages of the disclosed embodiments are further discussed below.

Before describing these aspects of the embodiments in detail, however, it is first desirable to describe examples of devices that can be present within, for example, a system for determining movement variability, illness and injury risk and recovery readiness based on human movement data, as well as examples of their operation, all of which can be used with the embodiments described herein.

### Example Embodiment of Systems for Determining Movement Variability, Illness and Injury Risk and Recovery Readiness

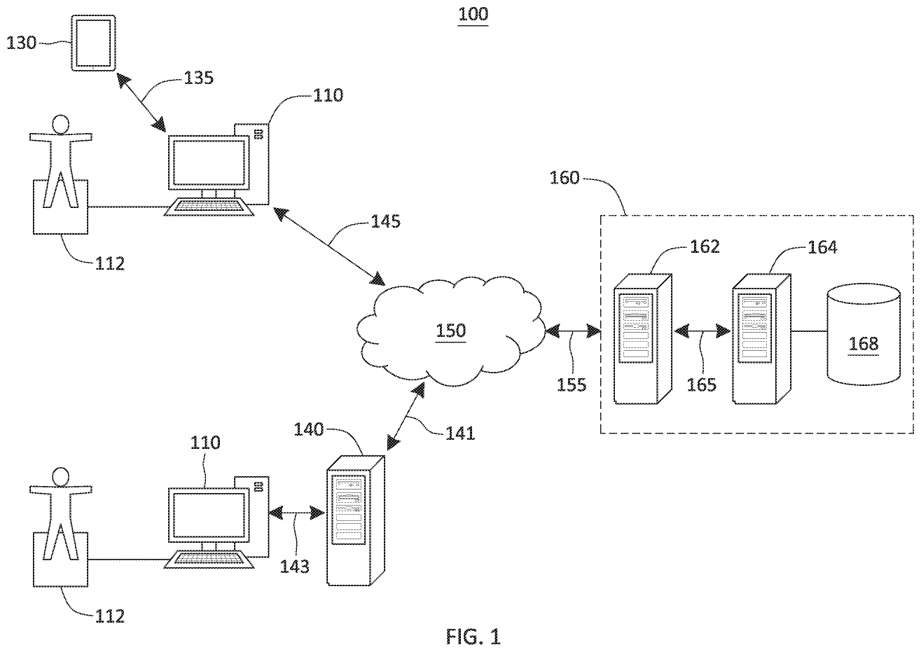

FIG. 1 is a conceptual diagram depicting an example embodiment of a system 100 for determining movement variability, illness and injury prediction and recovery readiness based at least in part on postural balance data, and which can be used with the embodiments of the present disclosure. System 100 includes a remote server system 160 configured to receive data from one or more computing devices 110, and which can comprise a front-end server 162 for interfacing with said computing devices 110, and a back-end server 164 that interfaces with both the front-end server 162 and database 168. Remote server system 160 can be a location-independent server system (e.g., cloud-based), which is accessible by a variety of computing devices 110 in geographically dispersed locations. Front-end server 162 can be in communication with back-end server 164 over a local area network, and can also communicate with computing devices 110 over communication path 155, which can include wired or wireless communications over network 150. In many of the embodiments disclosed herein, network 150 can be the Internet. In other embodiments, however, network 150 can also comprise one or more of an intranet, a wide area network, a local area network, a metropolitan area network, a virtual private network, a cellular network, or any other type of wired or wireless network. Furthermore, although front-end server 162 and back-end server 164 are depicted in FIG. 1 as two discrete devices, those of ordinary skill in the art will recognize that the functionalities and services of those devices can be implemented on a single centralized device or, in the alternative, can be distributed across multiple discrete devices in geographically dispersed locations. Likewise, those of skill in the art will recognize that the representations of servers in the embodiments disclosed herein, and as shown in FIG. 1, are intended to cover both physical servers and virtual servers (or “virtual machines”).

Referring still to FIG. 1, one or more local computing devices 110 are provided for receiving sensor data from sensor device 112, processing and extracting values from sensor data, and transmitting processed sensor data over network 150 to remote server system 160. Local computing device 110 can be a personal computer, laptop computer, wearable computer, desktop computer, workstation computer, or any other similar computing device, each of which can be communicatively coupled to a sensor device 112, which is configured to sense one or more movements (e.g., balance movements) performed by a user. Sensor device 112 can be connected to local computing device 110 via a wired or wireless communication link. Additionally, as shown in FIG. 1, a mobile computing device 130, such as a tablet computer, laptop, smart phone, or wearable computing device, can also be communicatively coupled to local computing device 110 through a wired or wireless communication link. Mobile computing device 130 can be configured to send and receive data to and from sensor device 112 via computing device 110 through communication path 135. In other embodiments, however, mobile computing device 130 can be configured to communicate directly with sensor device 112 through Bluetooth, Bluetooth Low Energy, 802.11x, UHF, NFC or any other standard wireless communications protocol. In some of the embodiments, mobile computing device 130 is configured to operate according to a mobile operating system such as Android and/or iOS. Local computing device 110 can be configured to transmit and receive data over communication path 145 through network 150, which, as described earlier, can comprise the Internet, an intranet, a wide area network, a local area network, a metropolitan area network, a virtual private network, a cellular network, or any other type of wired or wireless network.

In some embodiments, a local server system 140 can reside on the same local area network as local computing device 110. Local server system 140 can receive and store processed sensor data from local computing device 110, and in turn, transmit locally stored sway velocity and velocity frequencies scores, illness and injury risk scores, readiness scores, and other normalized scores to local computing device 110 over communications path 143. Local server system 140 can also synchronize a local database with the database 168 of the remote server system 160. In this regard, local server system 140 can serve as a proxy or intermediary between local computing device 110 and remote server system 160. In certain instances, this topology may be preferable, such as where heightened security is needed for local computing device 110 and/or the local area network on which local computing device 110 and local server system 140 reside. For example, the owner of local computing device 110 may not want to permit any or some of the processed sensor data collected through local computing device 110 to be transmitted to the remote server system 168, which may be shared by multiple tenants. In other instances, this topology may be preferable, for example where computing performance can be improved if sensor data can be processed locally at the local server system 140. Under those circumstances, local server system 140 can serve as a gateway, and conduct one-way synchronization or selective synchronization of the local database with database 168 of remote server system 160.

### Example Embodiment of Local Computing Device

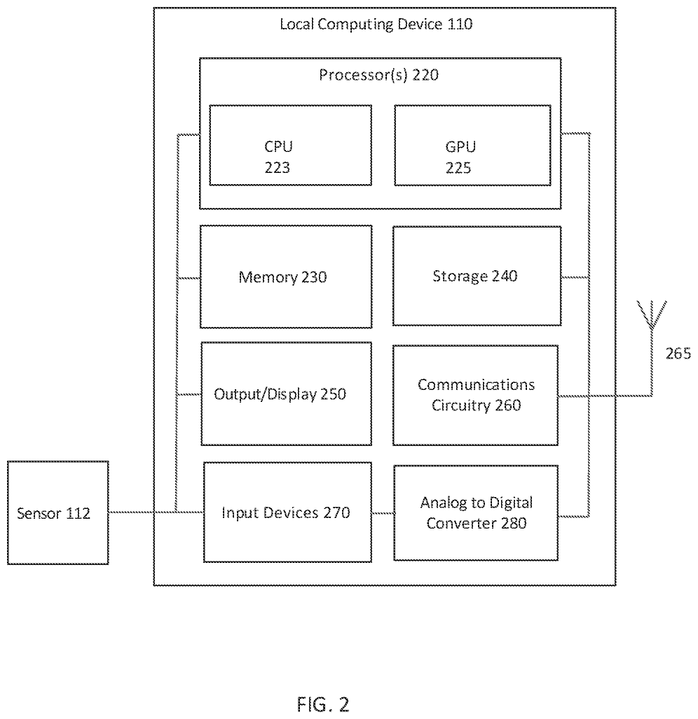

FIG. 2 is a block diagram depicting an example embodiment of local computing device 110. Local computing device 110 can include one or more processors 220, which can comprise, for example, one or more of a general-purpose central processing unit (“CPU”), a graphics processing unit (“GPU”), an application-specific integrated circuit (“ASIC”), a field programmable gate array (“FPGA”), an Application-specific Standard Products (“ASSPs”), Systems-on-a-Chip (“SOCs”), Programmable Logic Devices (“PLDs”), or other similar components. Processors 220 can comprise one or more processors, microprocessors, controllers, and/or microcontrollers, or a combination thereof, wherein each component can be a discrete chip or distributed amongst (and a portion of) a number of different chips, and collectively, can have the majority of the processing capability for acquiring, validating and analyzing user movement data. Local computing device 110 can also include memory 230, which can comprise non-transitory memory, RAM, Flash or other types of memory. Furthermore, local computing device 110 can include one or more mass storage devices 240, an output/display component 250, communications circuitry 260, which can include one or more wireless and/or wired network interfaces, an antenna 265 coupled to communications circuitry 260, an analog to digital converter component 280 configured to convert an analog signal received from a sensor device into a digital signal, and an input device component 270, which can include keyboards, mice, trackpads, touchpads, microphones and other user input devices, each of which can be communicatively coupled to local computing device 110 via a wired or wireless connection.

In many of the embodiments of the present disclosure, input devices component 270 can also include a sensor device 112, which can comprise one or more sensors configured to sense various characteristics of a user movement, including movement variability. In many embodiments, for example, sensor device 112 can comprise a force plate including one or more piezoelectric sensors within a single housing, wherein the one or more piezoelectric sensors are adapted to measure ground reaction forces while one or more balance movements are performed by a user. In some embodiments, sensor device 112 can comprise a force plate including one or more strain gauge sensors within a single housing. In still other embodiments, sensor device 112 can include multiple types of sensors, in which data received from a first type of sensor can be used to corroborate the data received from a second type of sensor. For example, sensor device 112 can comprise a force plate including one or more piezoelectric sensors, as described earlier, and additionally, one or more accelerometers embedded within a portion of a user's footwear. Sensor data from the piezoelectric sensors and the accelerometers can be correlated, time synchronized and/or multiplexed by local computing device 110 to determine and corroborate various characteristics of the one or more balance movements performed by the user. As understood by those of skill in the art, the aforementioned components are electrically and communicatively coupled in a manner to make one or more functional devices.

In some embodiments, sensor device 112 may include one or more sensors worn on the body of the user.

Referring still to FIG. 2, communications circuitry 260 of local computing device 110 can be configured to communicate directly with remote server system 160, or via local server system 140. In many of the embodiments disclosed herein, local computing device 110 is configured to receive sensor data generated by sensor device 112 in response to a user performing one or more balance tests. The received sensor data can be processed and transmitted to remote server system 160 which, in turn, transmits one or more scores, such as sway velocity and velocity frequencies scores, illness and injury risk scores, readiness scores, or one or more normalized scores correlating to the balancing tests performed by the user to the local computing device 110. In some embodiments, the received sensor data can be processed at the local server system 140 and synchronized with the remote server system 160. In many of the embodiments disclosed herein, the scores can be visually displayed through a user interface on local computing device 110. In some embodiments, for example, the scores can be T-scores that are visually displayed in an easy-to-read format, such as a vertical bar chart. In other embodiments, normalized scores can be depicted as a plotted line as a function of time. These graphical user interfaces, as well as other visual representations, can be generated by processors 220 in response to instructions, e.g., in the form of a locally installed application, which reside in memory 230 of local computing device 110.

As described earlier, local computing device 110 is represented in FIG. 2 as a personal computer, desktop computer, laptop computer or workstation. However, in some embodiments, the one or more local computing devices 110 can also include laptop computers, tablet computing devices, smartphones, personal digital assistants, wearable computing devices or other mobile computing devices.

Example Embodiments of Remote Server System

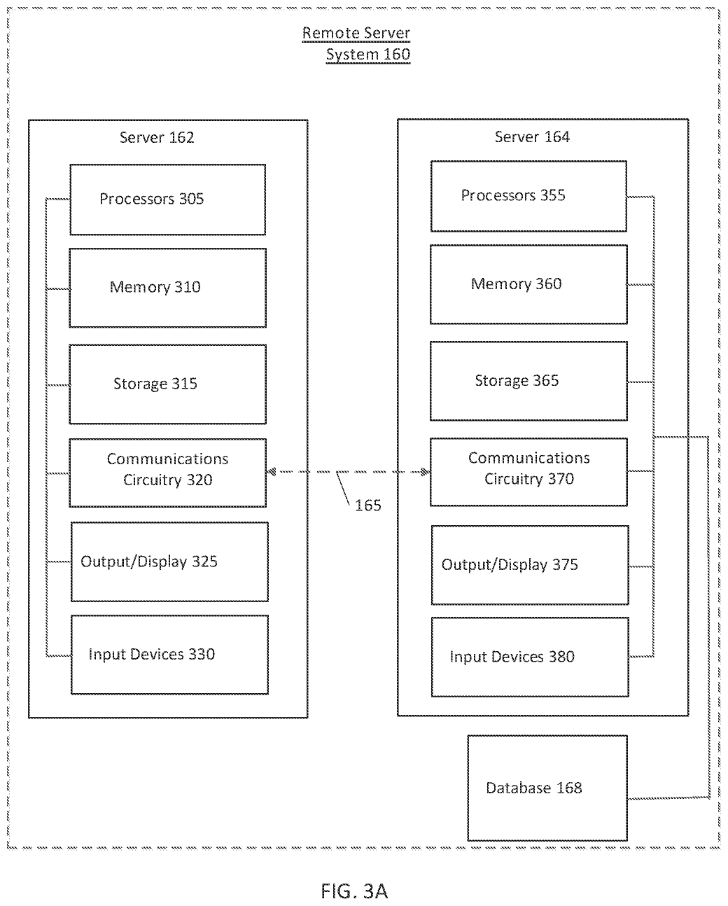

FIG. 3A is a block diagram depicting an example embodiment of remote server system 160 comprising one or more servers, and which can include a front-end server 162 and a back-end server 164. As shown in the diagram, servers 162, 164 can each include, respectively, an output/display component (325, 375), one or more processors (305, 355), memory (310, 360), including non-transitory memory, RAM, Flash or other types of memory, communications circuitry (320, 370), which can include both wireless and wired network interfaces, mass storage devices (315, 365), and input devices (330, 380), which can include keyboards, mice, trackpads, touchpads, microphones, and other user input devices. The one or more processors (305, 355) can include, for example, a general-purpose CPU, a GPU, an ASIC, an FPGA, ASSPs, SOCs, PLDs, and other similar components, and furthermore, can comprise one or more processors, microprocessors, controllers, and/or microcontrollers, each of which can be a discrete chip or distributed amongst (and a portion of) a number of different chips. As understood by one of skill in the art, these components are electrically and communicatively coupled in a manner to make a functional device.

In some embodiments, front-end server 162 can be configured such that communications circuitry 320 provides for a single network interface which allows front-end server 162 to communicate with the one or more local computing devices, as well as back-end server 164. In other embodiments, front-end server 162 can be configured such that communications circuitry 320 provides for two discrete network interfaces to provide for enhanced security, monitoring and traffic shaping and management. In addition, in most embodiments, front-end server 162 includes instructions stored in memory 310 that, when executed by the one or more processors 305, cause the one or more processors 305 to receive processed sensor data from one or more local computing devices, store processed sensor data to a database 168, and generate and transmit one or more scores associated with a user movement to a local computing device. In addition, the instructions stored in memory can further cause the one or more processors to perform one or more of the following routines: aggregate processed sensor data by various categories including by gender, by age, by body weight, by preferred sport and/or by position within a preferred sport; generate and store normalized scores associated with a user movement for one or more of the aforementioned categories; update scores based on newly received processed sensor data from the one or more local computing devices; and perform synchronization between database 168 and one or more databases residing on local server systems.

Referring still to FIG. 3A, server 164 can include database 168 for storing processed sensor data indicative of one or more characteristics of a user movement. In some embodiments, database 168 can reside on back-end server 164. In other embodiments, database 168 can be distributed or be part of a storage area network, for example, to which back-end server 164 is communicatively coupled. Back-end server 164 can also include communications circuitry 370 which can be configured to facilitate communications to and from front-end server 162. Similar to the configuration of front-end server 162, in many embodiments, communications circuitry 370 can include a single network interface, either wired or wireless; or, in other embodiments, communications circuitry 370 can include multiple network interfaces, either wired or wireless, to provide for enhanced security, monitoring and traffic shaping and management.

### Example Embodiments of Methods for Determining Movement Variability, Illness and Injury Risk and Recovery Readiness

Described herein are example embodiments of methods and systems for determining illness and injury risk of a user based on user movement data, including movement variability data.

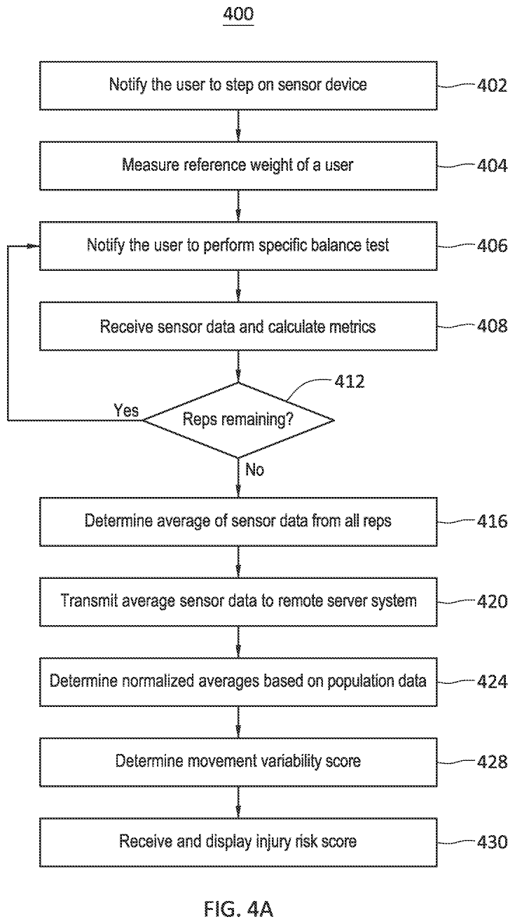

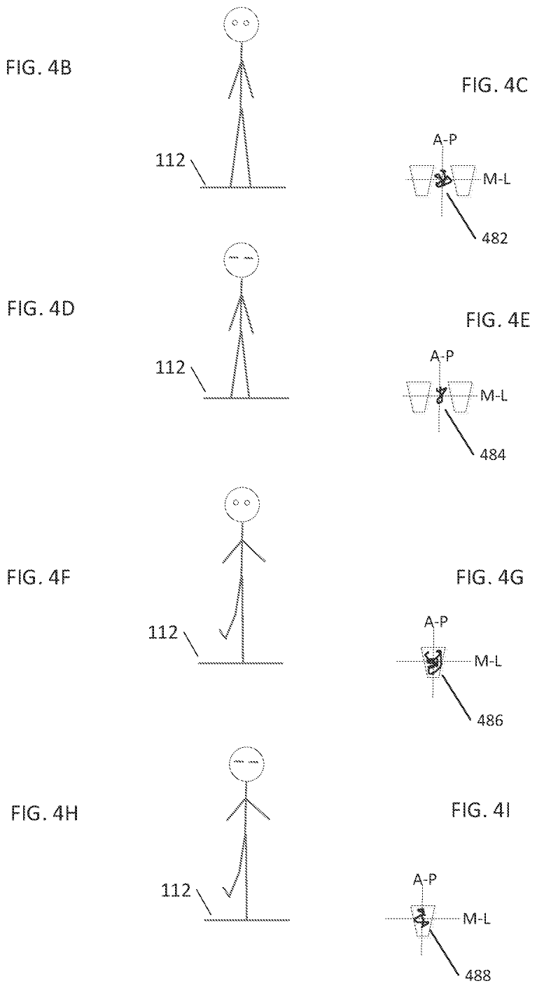

FIG. 4A is a flow diagram depicting an overview of an example embodiment of a method 400 for determining a user's movement variability, illness and injury risk and recovery readiness scores based on user movement data, including center of foot pressure data. Those of skill in the art will understand that the method steps disclosed herein can comprise instructions stored in memory of the local or mobile computing device, and that the instructions, when executed by the one or more processors of the local or mobile computing device, can cause the one or more processors to perform the steps disclosed herein.

As shown at the top of FIG. 4A, the method may include instructing a user to step on to the sensor device at Step 402. At Step 404, the user's reference weight is measured. In some embodiments, the reference weight can be inputted manually by the user or another person. At Step 406, a visual or audio notification is outputted by the local or mobile computing device instructing the user to perform a balance task, for example, stand with two feet on the sensor device with eyes open, stand with two feet on the sensor device with eyes closed, stand on one foot (left or right) on the sensor device with eyes open, stand on one foot (left or right) on the sensor device with eyes closed (as shown in FIGS. 4B to 4I).

At Step 408, while the user is in the requested position, the computing device receives sensor data from the sensor device, wherein the sensor data is indicative of the force generated by the user as a function of time. In some embodiments, the sensor data include at least these measurements: (1) sway velocity and (2) sway velocity frequencies These measurements are based at least in part from reading of movement of the center of the foot pressure (as shown by 482, 484, 486 and 488 in FIGS. 4B to 41). In some embodiments, the measurements of force may be captured, for example, every millisecond for the duration of a scan. These time-ordered sequences of raw measurements may be referred to as time series data. In some embodiments, the system may receive one segment of sensor data for all sensors, then convert the data into time series for vertical force and center of pressure in two directions. The system may calculate metrics for the segment. The metrics may include, for example, average velocity of center of pressure (sway velocity), multi-scale sample entropy of each time series, vector coding calculation, and medial lateral weight shift.

At Step 412, if it is determined that additional repetitions are required, the method returns to Step 406, and a visual or audio notification is outputted by the local or mobile computing device instructing the user to perform another repetition of the balance task. In some embodiments, a rest period can be interposed after Step 412, during which the user can rest and recover from the previous task for a short period of time (e.g., 10 seconds) before being notified to perform the test again at Step 406. In some other embodiments, six repetitions of the same task (test) are required. Those of skill in the art will appreciate that this number of repetitions is not meant to be exhaustive, and that other numbers of repetitions are fully within the scope of the present disclosure.

If it is determined that no repetitions are remaining then, at Step 416, the local or mobile computing device determines an average of sensor data measurements acquired during the repetitions. For example, an average (1) sway velocity and (2) sway velocity frequencies value can be calculated for all repetitions. At Step 420, the averaged sensor data measurements are transmitted to the remote server system. In some embodiments, an authentication step can be interposed after Step 416, prior to transmission, in order to ensure that the local or mobile computing device is authorized to transmit data to the remote server system. In some embodiments, the authentication step can be manual, such as requiring the user to input a password at the local or mobile computing device. In other embodiments, the authentication step can be automated through a public or private key exchange.

Referring still to FIG. 4A, at Step 424, one or more average sensor data measurements can be normalized based at least in part on the sensor data measurements for a predetermined population of patients or athletes stored in a database residing on, or in communication with, the remote server system. In some embodiments, the predetermined population of patients or athletes can comprise the entire population of patients or athletes for which relevant data is stored in the database. In other embodiments, the predetermined population of patients or athletes can comprise a subset of the entire population. Subsets of patients or athletes can be categorized by gender, body weight range, age range, injury or illness type, and (for athletes) position within a preferred sport. Those of skill in the art will appreciate that these examples are not meant to be exhaustive, and that other subsets of population data within the database are fully within the scope of the present disclosure. In addition, in many of the embodiments of the present disclosure, the determination of the normalized values can be performed by the one or more processors of the remote server system by either of the front-end server or the back-end server. In other embodiments, however, the determination of the normalized values can be performed elsewhere, such as, for example, by a local server system (as shown in FIG. 1), or by the local or mobile computing device itself.

According to one aspect of the embodiments of the present disclosure, the normalized values can be T-scores. T-scores enable a user to take a raw value (e.g., the processed sensor data) and transform it into a standardized score that allows the user to contextualize her assessment within a population of relevant athletes. A standardized score is typically determined by using the mean and standard deviation values from the relevant population data, as represented by the following equation:

$$z = \frac{x - μ}{σ}$$

where z is the standard score, x is the raw score (i.e., processed sensor data), p is the mean of the relevant population, and a is the standard deviation of the relevant population. The T-score is a standard z score shifted and scaled to have a mean of 50 and a standard deviation of 10. A standard z score can be converted to a T-score by the following equation:

T=(z×10)+50

In this regard, T-scores are both meaningful and easy to comprehend. Unlike other standardized measures (e.g., z-scores), T-scores are always positive and typically comprise whole integers. In addition, a T-score of over 50 is above average, a T-score of below 50 is below average, and each increment of 10 represents one standard deviation away from the mean value.

Although shown as a positive number, the T-scores may also be negative.

At Step 428, one or more scores can be determined based at least in part on the normalized values.

In some embodiments, the one or more scores can be calculated using one or more learning machine algorithms.

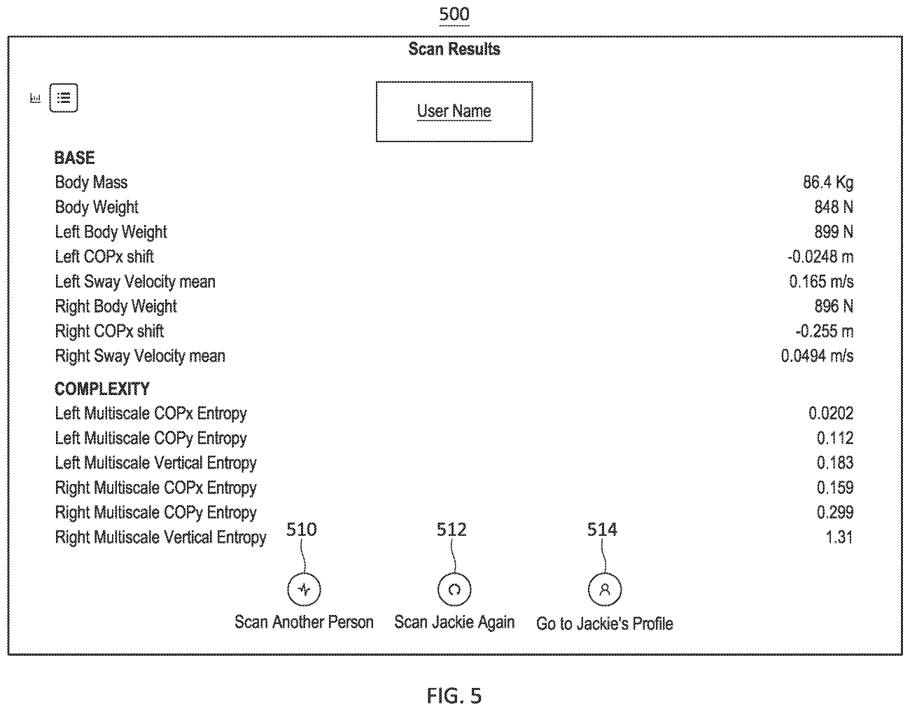

At Step 430, the normalized scores are received by the local or mobile computing device and can be displayed in a graphical user interface. FIG. 5 is an exemplary graphical user interface 500 illustrating results for a user that show exemplary metrics (features). In this example, the interface may include information received and calculated such as, the user's name and photo, body mass, weight, left body weight, left COPx (center of pressure along a mediolateral x-axis) shift, left sway velocity mean, right body weight, right COPx shift, and right sway velocity mean. The information displayed may also include, for example, multiscale sample entropy calculations, including left multiscale COPx entropy, left multiscale COPy (center of pressure along an anterior-posterior y-axis) entropy, left multiscale vertical entropy, right multiscale COPx entropy, right multiscale COPy entropy, and right multiscale vertical entropy.

As shown in the example of FIG. 5, the system may allow the user to perform another scan (option 512) or view the user's profile (option 514). Or the system may be set to scan another user (option 510).

In some embodiments, the data from the determination of movement variability, illness and injury risk and recovery readiness are stored in the database. The data can include one or more of the weight of the user at the time of assessment, the averaged sensor value measurements, the normalized values, the scores, date and time of the assessment, and other data as shown in FIG. 5.

In some embodiments, the remote server system may include complex statistical analysis using machine learning, for example, to predict illnesses or injuries, for example, concussion, to improve rehabilitation treatments, and to predict and determine recovery readiness. In these embodiments, the system may process time series data from the assessments, or scans, and extract one or more set of features from the time series data. The features may be extracted through a variety of techniques. Features may then be inputs for machine learning models, e.g., model to predict illness and injury risk. The techniques may include, for example:
Biophysics based analysis. In some embodiments, this technique may include estimating the average magnitude of the velocity of the center of pressure for a subject for a given period of time while balancing, for example, on one leg.
Statistics/signal processing based analysis. In some embodiments, this technique may include calculation of multiscale sample entropy for time series signals. The time series signals may include, for example, vertical force and center of pressure along coordinate axes.
Unsupervised learning techniques. In some embodiments, these techniques may include using an autoencoding temporal convolutional neural network to extract a set of features from segments of time series data.

In some embodiments, the system may include a trained injury risk model. In some embodiments, the model may be trained, using, for example, artificial neural network modelling techniques, to predict relative injury risk for individuals for a given set of features extracted from scans (e.g., a balance scan). In some embodiments, the models may be trained using injury event data associated with periods with which scan data is associated.

FIGS. 4B to 4I are pictorial diagrams depicting certain steps of methods 400, in which a user's movement variability, illness and injury risk and recovery readiness scores may be determined. FIG. 4B is a pictorial diagram showing the user standing on and staying stationary on the force plate 112 with both eyes open. FIG. 4C is a graph showing the movement 482 of the center of the pressure as the user in FIG. 4B stays stationary on the force place 112. The graphical representation 482 of the movement represents data sensed and recorded by the force plate 112. As shown, although the user is standing and staying stationary with both eyes open, there are still movements at the user's center balance, e.g., center of gravity. The graphical representation 482 shows body sway movements recorded at the center of the user's feet. In some embodiments, the changes in pressure from the feet on the force plate may be sensed, recorded, and measured. As described herein, the force plate may sense and record the movements as time series data, for example, at every millisecond.

FIG. 4D is a pictorial diagram showing the user standing on and staying stationary on the force plate 112 with both eyes closed. FIG. 4E is a graph showing the movement 484 of the center of the pressure as the user in FIG. 4D stays stationary on the force place 112. The graphical representation 484 of the movement represents data sensed and recorded by the force plate 112. As shown, although the user is standing and staying stationary with both eyes closed, there are still movements at the user's center of gravity. The graphical representation 484 shows body sway movements recorded at the center of the user's feet. In some embodiments, the changes in pressure from the feet on the force plate may be sensed, recorded, and measured. As described herein, the force plate may sense and record the movements as time series data, for example, at every millisecond.

FIG. 4F is a pictorial diagram showing the user standing on one leg and staying stationary on the force plate 112 with both eyes open. FIG. 4G is a graph showing the movement 486 of the center of the pressure as the user in FIG. 4F stays stationary on the force place 112. The graphical representation 486 of the movement represents data sensed and recorded by the force plate 112. As shown, as the user is standing and staying stationary on one leg with both eyes open, there are movements at the user's center of gravity (e.g., as the user loses balance and tries to gain and regain balance). The graphical representation 486 shows body sway movements recorded at the center of the user's foot. In some embodiments, the changes in pressure from the foot on the force plate may be sensed, recorded, and measured. As described herein, the force plate may sense and record the movements as time series data, for example, at every millisecond.

FIG. 4H is a pictorial diagram showing the user standing on one leg and staying stationary on the force plate 112 with both eyes closed. FIG. 41 is a graph showing the movement 488 of the center of the pressure as the user in FIG. 4H stays stationary on the force place 112. The graphical representation 488 of the movement represents data sensed and recorded by the force plate 112. As shown, as the user is standing and staying stationary on one leg with both eyes closed, there are movements at the user's center of gravity (e.g., as the user loses balance and tries to gain and regain balance). The graphical representation 488 shows body sway movements recorded at the center of the user's foot. In some embodiments, the changes in pressure from the foot on the force plate may be sensed, recorded, and measured. As described herein, the force plate may sense and record the movements as time series data, for example, at every millisecond.

Although FIGS. 4B to 4I depict specific standing positions, these positions are meant to be illustrative and non-exclusive. For example, the user in FIGS. 4F and 4H may stand on a different leg. Indeed, those of skill in the art will appreciate that other standing positions are fully within the scope of the present disclosure.

### Example Embodiments of Methods for Determining Recovery Readiness

In some embodiments, a recovery readiness score can indicate the ability of a patient to return to an activity or activities before an illness or an illness and injury. If the patient is an athlete the recovery readiness score can indicate the availability and ability of one or more users, e.g., an athlete or an athletic team, to participate in a sport on the day of the assessment. In some embodiments, the readiness score is determined based at least in part on the user's illness or injury risk score determined on the same day, movement variability score, and a frequency of assessments, or scans, the user has performed in the last predetermined length of time. A readiness for a group of users can be determined by averaging the readiness of each individual user on a particular day.

In some embodiments, a user's overall individual readiness score over time for a user can be determined by averaging historical readiness scores of the user.

In some embodiments, a group readiness score can be determined by averaging the readiness scores of all users in the group.

It should also be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1-21. (canceled)

22. A system for assessing a user's readiness, the system comprising:
measuring, by a computing device, a reference weight of a user;
notifying, by the computing device, the user to perform a balance test, wherein the balance test comprises standing in a stationary position on a force plate;
receiving, by the computing device, sensor data from the force plate during the balance test, wherein the sensor data comprises one or more center of pressure movement data over time;
determining, the computing device, one or more averages of the one or more center of pressure movement data;
transmitting, by the computing device, the one or more averages to a server system coupled to the computing device;
normalizing, by the server system, the one or more averages based on a database residing on or in communication with the server system;
determining, by the server system, a movement variability score based on the one or more normalized averages;
determining, by the server system, an illness and injury risk score based on the one or more normalized averages;
determining, by the server system, a frequency of assessments of the user;
determining, by the server system, a readiness score based at least in part on the movement variability score, the illness and injury risk score, the frequency of assessments; and
receiving, by the computing device, from the server system and displaying the readiness score.

23. The system of claim 22, wherein the balance test comprises standing in the stationary position on the force plate with the user's both feet being on the force plate and the user's both eyes open.

24. The system of claim 22, wherein the balance test comprises standing in the stationary position on the force plate with the user's both feet being on the force plate and the user's both eyes closed.

25. The system of claim 22, wherein the balance test comprises standing in the stationary position on the force plate with only a first foot of the user's feet being on the force plate and the user's both eyes open.

26. The system of claim 22, wherein the balance test comprises standing in the stationary position on the force plate with only a first foot of the user's feet being on the force plate and the user's both eyes closed.

27. The system of claim 22, wherein the one or more center of pressure movement data includes sway velocity and sway velocity frequencies.

28. The system of claim 22, wherein the steps of notifying the user to perform the balance test and receiving sensor data are repeated a plurality of times.

29. The system of claim 22, wherein the step of determining the one or more averages comprises averaging each of the one or more force measurements across the plurality of repetitions.

30. The system of claim 22, wherein the step of normalizing the one or more averages based on a database having a predetermined population of users.

31. (canceled)

32. The system of claim 30, wherein the predetermined population of users comprises a subset of the population of users.

33. The system of claim 32, wherein the subset is categorized by at least one of gender, body weight range, age range, injury or illness type, and position within a preferred sport.

34. The system of claim 22, wherein the step of determining a movement variability score is further based on a machine learning model.

35. The system of claim 22 further comprising performing, by the server system, statistical analysis to predict illnesses or injuries.

36. The system of claim 35 further comprising performing, by the server system, complex statistical analysis to predict recovery readiness.

37. The system of claim 36, wherein the complex statistical analysis includes using machine learning.

38. The system of claim 22 further extracting, by the server system, one or more set of features from the sensor data.

39. The system of claim 38, wherein the step of extracting is done through one of a biophysics based analysis, a statistics/signal processing based analysis, and an unsupervised learning technique.

40. The system of claim 39, wherein the biophysics based analysis includes estimating the average magnitude of a velocity of the one or more center of pressure for the user for a given period of time while balancing.

41. The system of claim 39, wherein the statistics/signal processing based analysis includes calculation of multiscale sample entropy for sensor data over a given time.

42. The system of claim 39, wherein the unsupervised learning technique includes using an autoencoding temporal convolutional neural network.
